(19) United States
(12) Patent Application Publication
(10) Pub. No.: US 2007/0180562 A1
(43) Pub. Date: Aug. 2, 2007

(54) Method of transforming plants

(75) Inventors: John E. Thompson, Waterloo (CA); Tzann-Wei Michael Wang, Waterloo (CA); Wendy Wu, Waterloo (CA)

Correspondence Address: KENYON & KENYON LLP, 1500 K STREET N.W., SUITE 700, WASHINGTON, DC, 20005, US

(73) Assignee: Senesco Technologies, Inc., New Brunswick, NJ (US)

(21) Appl. No.: 11/412,962

(22) Filed: Apr. 28, 2006

Related U.S. Application Data
(60) Provisional application No. 60/690,160, filed on Jun. 14, 2005.

(51) Int. Cl. A01H 5/00; C12N 15/82

(52) U.S. Cl. 800/278; 800/281; 800/306; 554/8

(57) ABSTRACT

The present invention relates to novel method of vacuum infiltration transformation of plants which provides an increased efficiency of transformation over previously known vacuum infiltration methods, as well as seeds, plants and plant parts transformed with the methods.

This application claims priority to and incorporates by reference in its entirety, provisional application 60/690,160, filed on Jun. 14, 2005.

### FIELD OF THE INVENTION

The present invention relates to novel method of transforming plants which provides an increased efficiency of transformation over previously known methods.

### BACKGROUND

Current transformation technology provides an opportunity to engineer plants with desired traits. Major advances in plant transformation have occurred over the last few years. Plant transformation methods include direct co-cultivation of plants, tissues or cells with Agrobacterium tumefaciens or direct infection (Miki, et al, Meth. in Plant Mol. Biol. and Biotechnology, (1993), p. 67-88); direct gene transfer into protoplasts or protoplast uptake (Paszkowski, et al., EMBO J., 12:2717 (1984); electroporation (Fromm, et al., Nature, 319:719 (1986); particle bombardment (Klein et al., BioTechnology, 6:559-563 (1988); injection into meristematic tissues of seedlings and plants (De LaPena, et al., Nature, 325:274-276 (1987); injection into protoplasts of cultured cells and tissues (Reich, et al., BioTechnology, 4:1001-1004 (1986)) and vacuum infiltration.

However, in many major crop plants, serious genotype limitations still exist. Transformation of some agronomically important crop plants continues to be both difficult and time consuming. Often the transformation efficiencies are low. The major technical challenge facing plant transformation biology is the development of methods and constructs to produce a high proportion of plants showing predictable transgene expression without collateral genetic damage.

There thus remains a need for an improved method of transforming plants that provides a better transformation efficiency. The present invention meets this need.

### SUMMARY OF THE INVENTION

The present invention provides a method for increasing transformation efficiency of vacuum infiltration transformation of a plant. This method comprises vacuum infiltration of a plant with a desired nucleic acid. New flowers that formed on the plant after vacuum infiltration (and thus, were not transformed by the vacuum infiltration), are removed. Removal of the new flowers enhances the proportion of transformed seed and thus increases transformation efficiency.

Preferably transformation efficiency is increased over prior vacuum infiltration methods. Preferably transformation efficiency is at least 10%. More preferably transformation efficiency is at least 20%, 30%, 40%, 50% or 60% or any range greater than obtained from previously known vacuum infiltration methods.

Any plant may be transformed in this method, including any monocot or dicot, including crop plants known to have low transformation efficiencies such as, but not limited to corn, rice, soybean, alfalfa, and canola.

The present invention also provides a method for increasing transformation efficiency of a vacuum infiltration transformation of a plant comprising transforming a plant with a desired nucleic acid by vacuum infiltration; allowing new flowers to form on the plant and performing a second vacuum infiltration on the plant after the formation of new flowers. The second vacuum infiltration on the plant after the formation of new flowers increases transformation efficiency as in enhances the proportion of transformed seed.

The present invention also provides a transformed seed produced by the methods of the present invention and also includes a plant or plant part produced from the transformed seed.

### BRIEF DESCRIPTION OF THE FIGURES

FIGS. 1A-1B provide canola DHS amino acid and polynucleotide sequences.

FIG. 2 provides canola DHS and the construction of pKYLX71-sense DHS.

FIG. 3 shows in bar graph form that inhibition of DHS expression increases seed yield in canola.

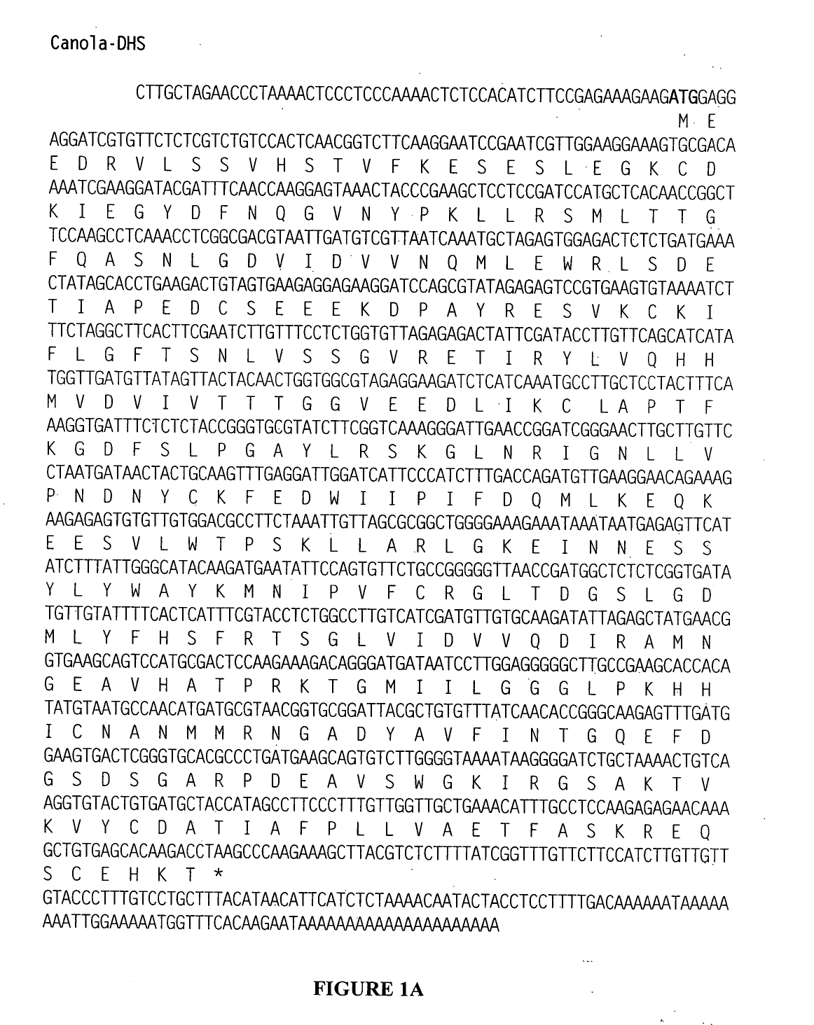

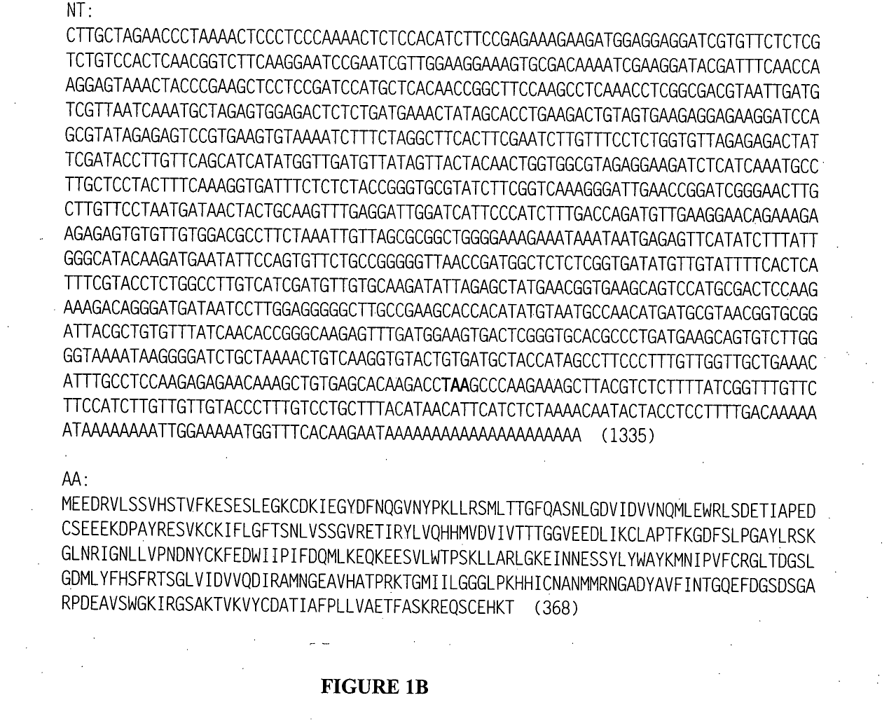

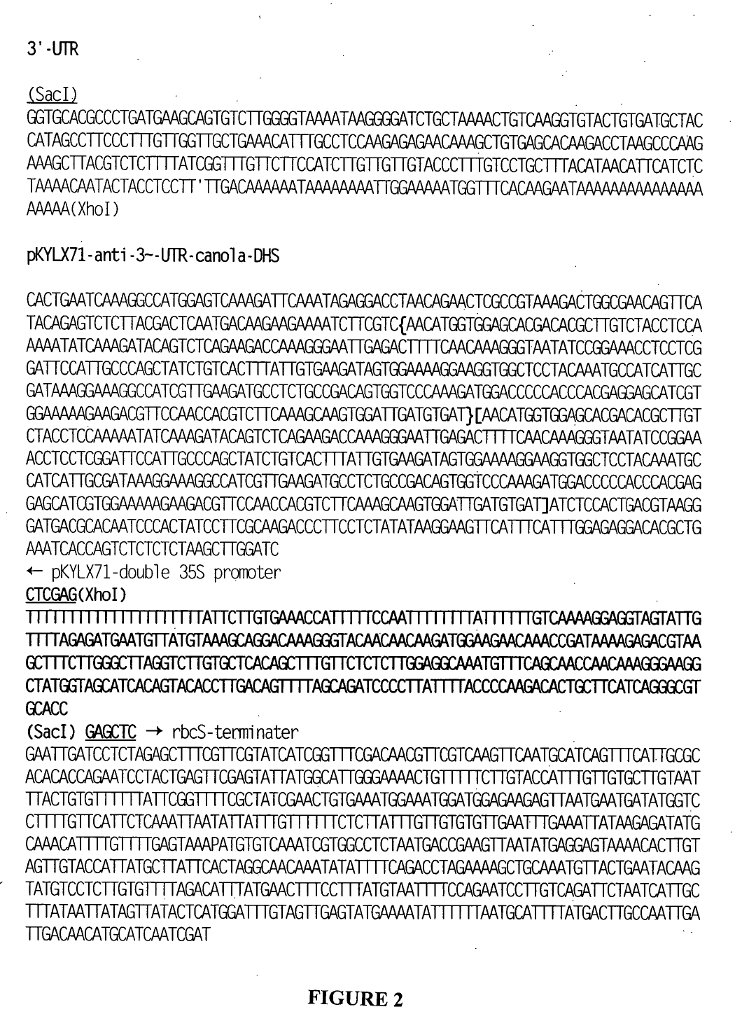

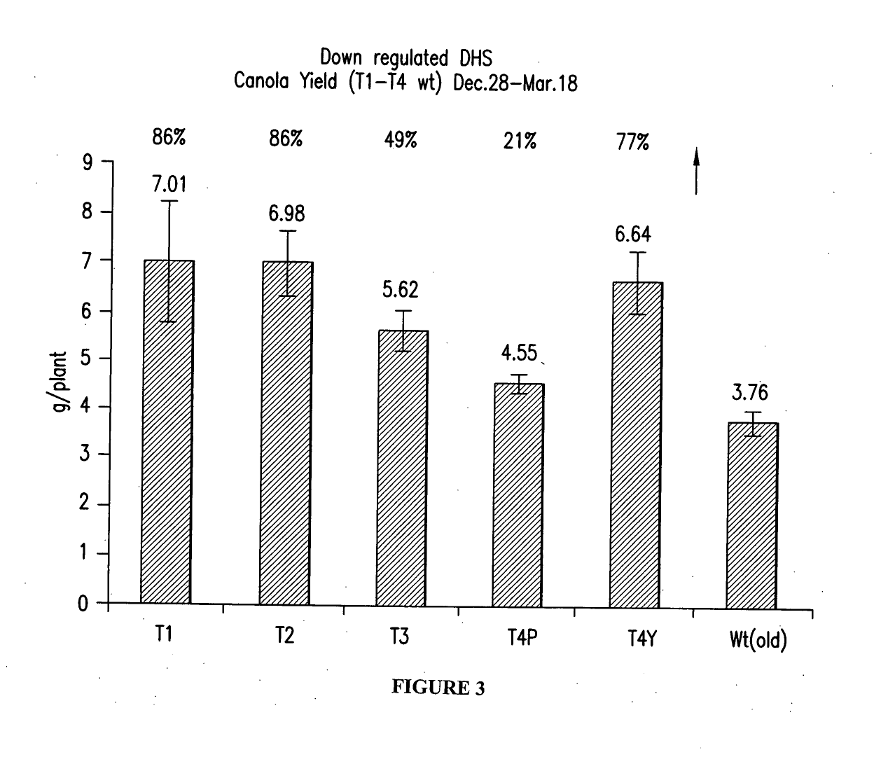

### DETAILED DESCRIPTION OF THE INVENTION

The present invention provides a method for increasing transformation efficiency of vacuum infiltration transformation of a plant. This method comprises vacuum infiltration of a plant with a desired nucleic acid. Vacuum infiltration methods are known and have been reported on canola plants by Wang W C, Menon G, Hansen G (2003) “Development of novel Agrobacterium-mediated transformation method to recover transgenic Brassica napus plants” Plant Cell Rep 22: 274-281. However, the efficiency of transformation was only up to 0.18%. The present invention provides for an increased efficiency of transformation and the present inventors have routinely obtained transformation rates of 50-60%. Preferably, transformation efficiency is increased over prior vacuum infiltration methods, that is the number of transformed seeds as compared to non transformed seeds in increased over the numbers one would get using previously known vacuum infiltration methods. Preferably transformation efficiency is at least 10% (i.e., 10% of the seed is transformed). More preferably transformation efficiency is at least 20%, 30%, 40%, 50% or 60% or any range greater than obtained from previously known vacuum infiltration methods.

In the vacuum infiltration, it is preferred to induce early bolting and flowering to ensure that the entire plant is not too large nor has too long of flower stalks to allow the pant to fit into a bell jar for vacuum infiltration. Preferably the inflorescences are vacuum-infiltrated infiltrated with Agrobacterium tumefaciens expressing the desired nucleic acid (i.e. a transgene). The vacuum infiltration is performed as described for Arabidopsis in Bechtold N, Ellis J, Pelletier G (1993), “In planta Agrobacterium-mediated gene transfer by infiltration of adult Arabidopsis thaliana plants,” C R Acad Sci Paris Life Sci 316: 1194-1199. For example, a single colony of an Agrobacterium tumefaciens culture is transformed with a desired nucleic acid under the regulation of a desired promoter and grown in a suitable broth to a desired OD₆₀₀ₙₘ, such as greater than 2.0. The bacterial cells are pelleted and resuspended in a suitable buffer known in the art such as modified 0.5X MS media solution [Per litre: 2.2 g Murashige and Skoog Basal Medium, 50 g sucrose, 0.5×g MES (2-morpholinoethanesulfonic acid), pH 5.7, 10 μl of benzylaminopurine (10 mg/ml) and 100 μl VAC-IN-STUFF (Silwet L-77, OSi Specialties Inc.,)]. The Agrobacterium solution is then placed inside of a bell jar attached to a vacuum/pressure pump, and the flowering portion of a bolting plant is dipped into the Agrobacterium solution. Often times the bolt/plant may have to be manipulated to fit the entire plant into the bell jar. The vacuum/pressure pump is turned on, and vacuum is applied to the bell jar for a sufficient time, such as 15 minutes. The pump is turned off, and the vacuum is slowly released to ambient pressure. Excess Agrobacterium solution may be blotted off of the plant, and the plant returned to the greenhouse to complete its growing cycle and to allow the flowers to develop seed.

In the present method, new flowers that have formed after the plants have been vacuum-infiltrated are removed to enhance the proportion of transformed seed. To assist in the identification of the new flowers, the bolt or plant may be marked at the time of vacuum infiltration and any growth or new flowers developing above the mark (and thus after the vacuum infiltration) are consider new flowers and thus were not exposed to the Agrobacterium solution in the vacuum infiltration. These new flowers thus would not develop into transformed seeds as they were never exposed to the transgene. By removing these new flowers and only allowing the flowers/inflorescences that were exposed to the transgene in the vacuum infiltration to develop into seed, the number of nontransformed seeds will decrease (thus enhancing the proportion of transformed seed and increasing transformation efficiency).

Transformed seed may be selected by any method known in the art. For example, the seeds may soaked in a selection media that corresponds to a selection marker used in the nucleic acid construct, such as, but not limited to kanamycin. For example, the harvested seeds may be surface sterilized in a solution of 1% sodium hypochlorite and 0.1% Tween-80, rinsing three times in sterile water and soaking in a solution of kanamycin (300 μg ml⁻1) for 30 min with slow mechanical rotation. The kanamycin-treated treated seeds are then planted in a suitable soil or soil substitute and then are maintained under greenhouse conditions. The seeds are germinated and selected for transformed seedlings. The seedlings are allowed to grow and develop seeds, which also contain the nucleic acid of choice (transformed seeds). Further generations may be developed to ensure stable transgenic seed lines.

In another embodiment, instead of removing the new flowers, a repeat vacuum infiltration may be performed to expose the new flowers to the Agrobacterium /nucleic acid construct. In fact, multiple infiltrations could be repeated on the new growths, the only limitation seeming to be the size of the plant able to fit into the bell jar.

Any plant may be transformed in this method, including any monocot or dicot, including crop plants known to have low transformation efficiencies such as, but not limited to corn, rice, soybean, alfalfa, and canola. Other plants include, but are not limited to fruit bearing plants such as apricots, apples, oranges, bananas, grapefruit, pears, tomatoes, strawberries, avocados, etc.; vegetables such as carrots, peas, lettuce, cabbage, turnips, potatoes, broccoli, asparagus, etc.; flowers such as carnations, roses, mums, etc.; agronomic crop plants such as corn, rice, soybean, alfalfa, canola and the like, and forest species such as deciduous trees, conifers, evergreens, etc. It may include plants of a variety of ploidy levels, including haploid, diploid, tetraploid and polyploid.

Transformed seed means a seed that has been transformed with a nucleic acid of choice, as compared to a non-transformed seed being a seed that has not been transformed with the nucleic acid of choice, e.g. a wild type seed. The nucleic acid may be any desired nucleic acid. By way of non-limiting examples, the nucleic acid may encode a heterologous gene not normally found in the plant's genome or it may encode a homologous gene (normally found in the plant's genome) to provide up-regulation with another “copy” of a gene normally found in the plant's genome. The nucleic acid may also be an antisense construct to provide for suppression of gene expression in the plant.

Another embodiment of the present invention provides for transformed seeds produced by the methods of the present invention as well as plants and plant parts grown/developed from the transformed seeds. One skilled in the art would understand the meaning of plant parts, which includes, but is not limited to, leaves, stalks, flowers, grains, oils, proteins, starches, seeds, pollen, etc.

### EXAMPLES

Canola seeds (B. napus cv. Westar) were germinated in Promix BX (Premier Brands, Red Hill, Pa.) in 6-inch pots. The pots were covered with saran wrap and maintained at 4° C. for 2 days after planting in order to induce early bolting and flowering. (Arteca NA (1996) Plant Growth Substances: Principles and Applications. Campman & Hall, New York, p 64 and Takahashi N, Phinney B O, MacMillan J (1991) Gibberellins. Spring-Verlag, New York, pp 362-363). Early bolting was important to ensure that the entire plant was not too large with flower stalks that were not too long, and could therefore fit into a bell jar for vacuum infiltration. The covers were then removed, and the pots were transferred to a greenhouse. Daylight in the greenhouse was approximately 16 h in duration, and the temperature ranged from 20 to 24° C. After 6-7 weeks, the inflorescences were vacuum-infiltrated with Agrobacterium tumefaciens expressing the transgene as described for Arabidopsis. (Bechtold N, Ellis J, Pelletier G (1993) In planta Agrobacterium-mediated gene transfer by infiltration of adult Arabidopsis thaliana plants. C R Acad Sci Paris Life Sci 316: 1194-1199). Specifically, a single colony of an Agrobacterium tumefaciens culture transformed with antisense 3'-UTR canola DHS cDNA under the regulation of the constitutive cauliflower mosaic virus 35S promoter was grown in 500 ml 2XYT broth overnight to an OD₆₀₀ₘₘ of greater than 2.0. The bacterial cells were pelleted by centrifugation at 3500 g at 4° C. and resuspended in a modified 0.5×MS media solution [Per litre: 2.2 g Murashige and Skoog Basal Medium, 50 g sucrose, 0.5 g MES (2-morpholinoethanesulfonic acid), pH 5.7, 10 μl of benzylaminopurine (10 mg/ml) and 100 μl VAC-IN-STUFF (Silwet L-77, OSi Specialties Inc.,)]. The Agrobacterium solution was placed inside of a bell jar attached to a vacuum/pressure pump, and the flowering portion of a bolting canola plant was dipped into the Agrobacterium solution. The bolt was gently twisted such that the whole plant fit inside the bell jar. The vacuum/pressure pump was turned on, and vacuum was applied to the bell jar for 15 minutes. The pump was turned off, and the vacuum was slowly released to ambient pressure. Excess Agrobacterium solution was blotted off of the canola plant, and the plant was returned to the greenhouse to complete its growing cycle. New flowers that formed after the plants had been vacuum-infiltrated were removed to enhance the proportion of transformed seed. Transformed seed was selected by first surface sterilizing the seeds harvested from vacuum-infiltrated plants in a solution of 1% sodium hypochlorite and 0.1% Tween-80, rinsing three times in sterile water and soaking in a solution of kanamycin (300 μg ml⁻¹) for 30 min with slow mechanical rotation. The kanamycin-treated seeds were then planted in Promix BX soil in 6- or 12-inch pots and maintained under greenhouse conditions. Transformed kanamycin-resistant seeds germinated within 2 days, and the fully expanded cotyledons of these seedlings were green. However, kanamycin-treated seeds harvested from vacuum-infiltrated plants that were not transformed as well as kanamycin-treated wild-type seed were distinguishable from transformed seed in that they did not germinate until 4 days after planting, and the cotyledons were yellow in colour. T4 seed for individual transgenic lines was obtained using this screen, and the transgenic traits segregated with the kanamycin-resistance phenotype for all generations.

Suppression of Deoxyhypusine Synthase Expression in Canola Increases Seed Yield

Deoxyhypusine synthase (DHS) mediates the first of two enzymatic reactions that convert inactive eukaryotic translation initiation factor-5A (eIF-5A) to an activated form able to facilitate translation. A full-length cDNA clone encoding canola (Brassica napus cv Westar) DHS was isolated from a cDNA expression library prepared from senescing leaves. DHS was suppressed in transgenic canola plants by expressing the antisense 3'-UTR of canola DHS cDNA under the regulation of the constitutive cauliflower mosaic virus (CaMV-35S) promoter. The transgenic canola plants were obtained by vacuum infiltration of canola inflorescences as described above. The transgenic plants had reduced levels of leaf DHS protein and exhibited delayed natural leaf senescence. Suppression of DHS also increased leaf size by 1.5- to 2-fold and resulted in increases in seed yield of up to 65%. This was attributable in part to an increase in the size of the siliques, which were on average 18% to 26% longer than wild-type siliques depending on the line. When wild-type and transgenic plants were grown in 6-inch pots, the increase in seed yield accruing from suppression of DHS was ˜4.5-fold greater than when the plants were grown in 12 inch pots. Thus suppression of DHS appears to ameliorate the effects of sub-lethal stress engendered by growth in small containers. The increase in seed yield for transgenic plants translates into a corresponding increase in seed oil content based on measurements of triacylglycerol, and there was no change in the fatty acid composition of the oil in transgenic seeds.

1. A method for increasing transformation efficiency of vacuum infiltration transformation of a plant comprising,
a) transforming a plant with a desired nucleic acid by vacuum infiltration; and
b) removing new flowers formed on said plant after said vacuum infiltration, wherein removing of the new flowers enhances the proportion of transformed seed, which in turn increases transformation efficiency.

2. The method of claim 1 wherein the increased efficiency results in a transformation efficiency of at least 10%.

3. The method of claim 2 wherein the increased efficiency results in a transformation efficiency of at least 30%.

4. The method of claim 3 wherein the increased efficiency results in a transformation efficiency of at least 40%.

5. The method of claim 3 wherein the increased efficiency results in a transformation efficiency of at least 50%.

6. The method of claim 1, wherein the plant is a crop plant selected from the group consisting of corn, rice, soybean, alfalfa, and canola.

7. The method of claim 1, wherein the plant is canola.

8. A method for increasing transformation efficiency of a vacuum infiltration transformation of a plant comprising,
a) transforming a plant with a desired nucleic acid by vacuum infiltration;
b) allowing new flowers to form;
c) performing a second vacuum infiltration on the plant after the formation of new flowers,
wherein the second vacuum infiltration on the plant after the formation of new flowers enhances the proportion of transformed seed, which in turn increases transformation efficiency.

9. A transformed seed produced by the method of claim 1.

10. A plant or plant part produced from the transformed seed of claim 9.

11. A canola plant produced by the method of claim 1.

12. Oil obtained from a canola plant produced by the method of claim 1.


SEQUENCE LISTING (6 sequences)

SEQ ID NO: 1 - DNA, 1335 bp, Brassica napus
  CDS (57)..(1160)
cttgctagaaccctaaaactccctcccaaaactctccacatcttccgagaaagaagatggaggaggatcgtgttctctcgtctgtccactcaacggtcttcaaggaatccgaatcgttggaaggaaagtgcgacaaaatcgaaggatacgatttcaaccaaggagtaaactacccgaagctcctccgatccatgctcacaaccggcttccaagcctcaaacctcggcgacgtaattgatgtcgttaatcaaatgctagagtggagactctctgatgaaactatagcacctgaagactgtagtgaagaggagaaggatccagcgtatagagagtccgtgaagtgtaaaatctttctaggcttcacttcgaatcttgtttcctctggtgttagagagactattcgataccttgttcagcatcatatggttgatgttatagttactacaactggtggcgtagaggaagatctcatcaaatgccttgctcctactttcaaaggtgatttctctctaccgggtgcgtatcttcggtcaaagggattgaaccggatcgggaacttgcttgttcctaatgataactactgcaagtttgaggattggatcattcccatctttgaccagatgttgaaggaacagaaagaagagagtgtgttgtggacgccttctaaattgttagcgcggctggggaaagaaataaataatgagagttcatatctttattgggcatacaagatgaatattccagtgttctgccgggggttaaccgatggctctctcggtgatatgttgtattttcactcatttcgtacctctggccttgtcatcgatgttgtgcaagatattagagctatgaacggtgaagcagtccatgcgactccaagaaagacagggatgataatccttggagggggcttgccgaagcaccacatatgtaatgccaacatgatgcgtaacggtgcggattacgctgtgtttatcaacaccgggcaagagtttgatggaagtgactcgggtgcacgccctgatgaagcagtgtcttggggtaaaataaggggatctgctaaaactgtcaaggtgtactgtgatgctaccatagccttccctttgttggttgctgaaacatttgcctccaagagagaacaaagctgtgagcacaagacctaagcccaagaaagcttacgtctcttttatcggtttgttcttccatcttgttgttgtaccctttgtcctgctttacataacattcatctctaaaacaatactacctccttttgacaaaaaataaaaaaaattggaaaaatggtttcacaagaataaaaaaaaaaaaaaaaaaaaa

SEQ ID NO: 2 - protein, 368 aa, Brassica napus
MEEDRVLSSVHSTVFKESESLEGKCDKIEGYDFNQGVNYPKLLRSMLTTGFQASNLGDVIDVVNQMLEWRLSDETIAPEDCSEEEKDPAYRESVKCKIFLGFTSNLVSSGVRETIRYLVQHHMVDVIVTTTGGVEEDLIKCLAPTFKGDFSLPGAYLRSKGLNRIGNLLVPNDNYCKFEDWIIPIFDQMLKEQKEESVLWTPSKLLARLGKEINNESSYLYWAYKMNIPVFCRGLTDGSLGDMLYFHSFRTSGLVIDVVQDIRAMNGEAVHATPRKTGMIILGGGLPKHHICNANMMRNGADYAVFINTGQEFDGSDSGARPDEAVSWGKIRGSAKTVKVYCDATIAFPLLVAETFASKREQSCEHKT

SEQ ID NO: 3 - DNA, 325 bp, Artificial Sequence
  Description of Artificial Sequence Synthetic construct
ggtgcacgccctgatgaagcagtgtcttggggtaaaataaggggatctgctaaaactgtcaaggtgtactgtgatgctaccatagccttccctttgttggttgctgaaacatttgcctccaagagagaacaaagctgtgagcacaagacctaagcccaagaaagcttacgtctcttttatcggtttgttcttccatcttgttgttgtaccctttgtcctgctttacataacattcatctctaaaacaatactacctccttttgacaaaaaataaaaaaaattggaaaaatggtttcacaagaataaaaaaaaaaaaaaaaaaaaa

SEQ ID NO: 4 - DNA, 906 bp, Artificial Sequence
  Description of Artificial Sequence Synthetic construct
cactgaatcaaaggccatggagtcaaagattcaaatagaggacctaacagaactcgccgtaaagactggcgaacagttcatacagagtctcttacgactcaatgacaagaagaaaatcttcgtcaacatggtggagcacgacacgcttgtctacctccaaaaatatcaaagatacagtctcagaagaccaaagggaattgagacttttcaacaaagggtaatatccggaaacctcctcggattccattgcccagctatctgtcactttattgtgaagatagtggaaaaggaaggtggctcctacaaatgccatcattgcgataaaggaaaggccatcgttgaagatgcctctgccgacagtggtcccaaagatggacccccacccacgaggagcatcgtggaaaaagaagacgttccaaccacgtcttcaaagcaagtggattgatgtgataacatggtggagcacgacacgcttgtctacctccaaaaatatcaaagatacagtctcagaagaccaaagggaattgagacttttcaacaaagggtaatatccggaaacctcctcggattccattgcccagctatctgtcactttattgtgaagatagtggaaaaggaaggtggctcctacaaatgccatcattgcgataaaggaaaggccatcgttgaagatgcctctgccgacagtggtcccaaagatggacccccacccacgaggagcatcgtggaaaaagaagacgttccaaccacgtcttcaaagcaagtggattgatgtgatatctccactgacgtaagggatgacgcacaatcccactatccttcgcaagacccttcctctatataaggaagttcatttcatttggagaggacacgctgaaatcaccagtctctctctaagcttggatc

SEQ ID NO: 5 - DNA, 325 bp, Artificial Sequence
  Description of Artificial Sequence Synthetic construct
tttttttttttttttttttttattcttgtgaaaccatttttccaattttttttattttttgtcaaaaggaggtagtattgttttagagatgaatgttatgtaaagcaggacaaagggtacaacaacaagatggaagaacaaaccgataaaagagacgtaagctttcttgggcttaggtcttgtgctcacagctttgttctctcttggaggcaaatgtttcagcaaccaacaaagggaaggctatggtagcatcacagtacaccttgacagttttagcagatccccttattttaccccaagacactgcttcatcagggcgtgcacc

SEQ ID NO: 6 - DNA, 662 bp, Artificial Sequence
  Description of Artificial Sequence Synthetic construct
gaattgatcctctagagctttcgttcgtatcatcggtttcgacaacgttcgtcaagttcaatgcatcagtttcattgcgcacacaccagaatcctactgagttcgagtattatggcattgggaaaactgtttttcttgtaccatttgttgtgcttgtaatttactgtgttttttattcggttttcgctatcgaactgtgcaaatggaaatggatggagaagagttaatgaatgatatggtccttttgttcattctcaaattaatattatttgttttttctcttatttgttgtgtgttgaatttgaaattataagagatatgcaaacattttgttttgagtaaaaatgtgtcaaatcgtggcctctaatgaccgaagttaatatgaggagtaaaacacttgtagttgtaccattatgcttattcactaggcaacaaatatattttcagacctagaaaagctgaaatgttactgaatacaagtatgtcctcttgtgttttagacatttatgaactttcctttatgtaattttccagaatccttgtcagattctaatcattgctttataattatagttatactcatggatttgtagttgagtatgaaaatattttttaatgcattttatgacttgccaattgattgacaacatgcatcaatcgat